(19) United States
(12) Patent Application Publication
(10) Pub. No.: US 2026/0000388 A1
(43) Pub. Date: Jan. 1, 2026

(54) SPECIMEN COLLECTION DEVICE

(71) Applicant: Quanta Vision Co., Ltd., New Taipei City (TW)

(72) Inventors: CHIH-YI HSU, New Taipei City (TW); YU-YU HO, New Taipei City (TW); WEI-KANG LIM, Selangor (MY); YA-CHIEH YU, New Taipei City (TW)

(21) Appl. No.: 19/252,617

(22) Filed: Jun. 27, 2025

(30) Foreign Application Priority Data
Jun. 28, 2024 (TW) 113206885

(51) Int. Cl. A61B 10/00; A61F 13/38

(52) U.S. Cl. CPC A61B 10/0051; A61F 13/38

(57) ABSTRACT

A specimen collection device, including: a tubular body, a first collection unit, a cap and a second collection unit. The tubular body includes an opening end portion, and the opening end portion includes an opening communicated with an interior of the tubular body, an inner periphery wall and an outer periphery wall. The inner periphery wall has a first connection portion, and the outer periphery wall has a second connection portion. The first collection unit is assembled to the opening end portion and has a third connection portion, an inlet and an outlet, the inlet is communicated with the outlet, and the outlet is communicated with the interior of the tubular body. The cap is assembled to the opening end portion and covers the opening, and the cap has a fourth connection portion. The second collection unit is insertable into the interior of the tubular body.

### BACKGROUND OF THE INVENTION

### Field of the Invention

The present invention relates to a specimen collection device.

### Description of the Prior Art

In the market, an increasing number of rapid test kits use saliva as the specimen. These test kits are primarily divided into two collection methods: spitting and swab-based sampling. The spitting method offers the advantage of being easy to operate, as it only requires the user to collect saliva from the oral cavity. However, if the saliva is too viscous or insufficient in quantity, it becomes difficult to collect and requires the test subject to precisely spit into the collection device, which can pose a challenge. On the other hand, for the swab-based method, the test subject must insert a swab into the inlet chamber for wiping, which may cause discomfort or aversion in some individuals.

However, commercially available products are limited to either spitting-type or swab-type collection devices. As a result, users must purchase and carry both types of specimen collection devices, leading to increased cost and inconvenience.

The present invention is, therefore, arisen to obviate or at least mitigate the above-mentioned disadvantages.

### SUMMARY OF THE INVENTION

The main object of the present invention is to provide a specimen collection device, which provides selectable specimen collection methods.

To achieve the above and other objects, the present invention provides a specimen collection device, including: a tubular body, including an opening end portion, the opening end portion including an opening communicated with an interior of the tubular body, an inner periphery wall and an outer periphery wall, the inner periphery wall having a first connection portion disposed thereon, the outer periphery wall having a second connection portion disposed thereon; a first collection unit, detachably assembled to the opening end portion, having a third connection portion, an inlet and an outlet, the inlet communicated with the outlet, the outlet communicated with the interior of the tubular body; and a cap, detachably assembled to the opening end portion and covering the opening, having a fourth connection portion disposed thereon; a second collection unit, being insertable into the interior of the tubular body; wherein one of the third connection portion and the fourth connection portion is detachably engageable with the first connection portion, and the other of the third connection portion and the fourth connection portion is detachably engageable with the second connection portion.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a preferable embodiment of the present invention;

FIG. 2 is a cross sectional view according to a preferable embodiment of the present invention when a first collection unit is assembled to a tubular body;

FIG. 3 is a cross sectional view according to a preferable embodiment of the present invention when a cap is assembled to the tubular body; and

FIG. 4 is a cross sectional view according to a preferable embodiment of the present invention when a second collection unit is used.

### DETAILED DESCRIPTION OF THE PREFERRED EMBODIMENTS

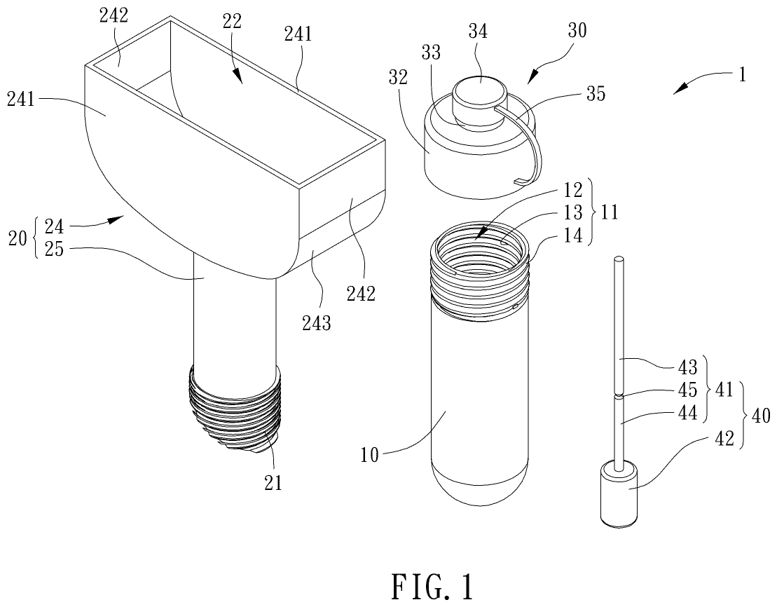

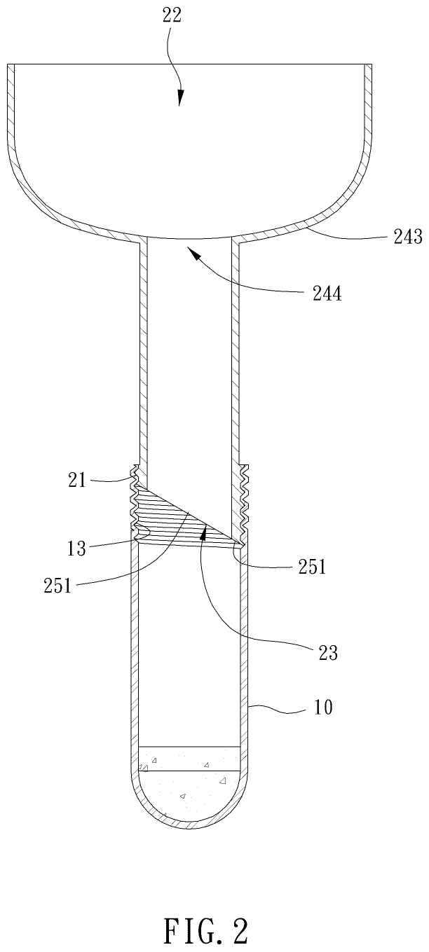

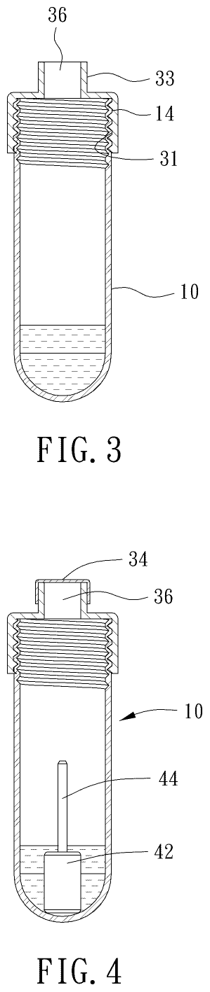

Please refer to FIGS. 1 to 4 for a preferable embodiment of the present invention. A specimen collection device 1 of the present invention includes a tubular body 10, a first collection unit 20, a cap 30 and a second collection unit 40.

The tubular body 10 includes an opening end portion 11, and the opening end portion 11 includes an opening 12 communicated with an interior of the tubular body 10, an inner periphery wall and an outer periphery wall. The inner periphery wall has a first connection portion 13 disposed thereon, and the outer periphery wall has a second connection portion 14 disposed thereon. The first collection unit 20 is detachably assembled to the opening end portion 11, the first collection unit 20 has a third connection portion 21, an inlet 22 and an outlet 23, and the inlet 22 is communicated with the outlet 23, and the outlet 23 is communicated with the interior of the tubular body 10. The cap 30 is detachably assembled to the opening end portion 11 and covers the opening 12, and the cap 30 has a fourth connection portion 31 disposed thereon. One of the third connection portion 21 and the fourth connection portion 31 is detachably engageable with the first connection portion 13, and the other of the third connection portion 21 and the fourth connection portion 31 is detachably engageable with the second connection portion 14.

The second collection unit 40 is insertable into the interior of the tubular body 10. The second collection unit 40 includes a rod 41 and a sampling member 42 disposed on the rod 41, and the sampling member 42 is made of a deformable absorbent material. The sampling member 42 may be formed of materials such as sponge, swab, or other liquid-absorbent substances. The sampling member 42 can absorb or retain a specimen (such as saliva or other liquid samples).

In this embodiment, the first collection unit 20 is funnel-shaped and further includes a receiving portion 24 and a guiding tube 25. The guiding tube 25 is connected with the receiving portion 24 and is communicated with an interior of the receiving portion 24, and an outer periphery of the guiding tube 25 has the third connection portion 21 disposed thereon. The guiding tube 25 detachably penetrates through the opening 12, and the third connection portion 21 is engageable with the first connection portion 13. Specifically, the third connection portion 21 is an external threaded structure, the first connection portion 13 is an internal threaded structure, and the external threaded structure is screwed with the internal threaded structure. Preferably, an end of the guiding tube 25 opposite to the receiving portion 24 includes a beveled surface 251, and the beveled surface 251 defines the outlet 23 so that the guiding tube 25 can be smoothly inserted into the opening 12.

An inner surface of the cap 30 has the fourth connection portion 31 disposed thereon, the cap 30 is sleeved to the opening end portion 11, and the fourth connection portion 31 is engageable with the second connection portion 14. The fourth connection portion 31 is an internal threaded section, the second connection portion 14 is an external threaded section, and the internal threaded section is screwed with the external threaded section.

Accordingly, by providing the first connection portion 13 and the second connection portion 14 on the inner and outer peripheral walls of the tubular body 10, respectively, the first collection unit 20 and the cap 30 having different radial dimensions can be assembled thereto, such that a fluid (such as a saliva sample, but not limited thereto) is less likely to leak from between the first connection portion 13 and the first collection unit 20, and between the fourth connection portion 31 and the cap 30. Moreover, different sampling options can be provided to user, such as using the first collection unit 20 to collect a specimen by spitting (e.g., saliva, but not limited thereto), or using the second collection unit 40 to collect a specimen with the sampling member 42 (e.g., saliva, but not limited thereto). The rod 41 includes a holding section 43 and a sampling section 44, a recessed section 45 is integrally connected between the holding section 43 and the sampling section 44 and recessed radially inward, and the sampling member 42 is disposed on the sampling section 44. Therefore, when the sampling section 44 is inserted into the tubular body 10, the holding section 43 and the sampling section 44 can be broken apart along the recessed section 45.

The first connection portion 13 and the second connection portion 14 correspond to each other in a radial direction of the tubular body 10. The receiving portion 24 includes two first flat surfaces 241 opposite to each other, two second flat surfaces 242 opposite to each other and an arcuate surface 243. Each of the two second flat surfaces 242 is arranged between the two first flat surfaces 241, and the arcuate surface 243 is connected between the two second flat surfaces 242 and has a through hole 244 disposed therethrough. The guiding tube 25 is connected to the arcuate surface 243 and communicated with the through hole 244 so that the saliva spit into the receiving portion 24 can flow along the arcuate surface 243 toward the through hole 244 without easily splashing, and the two first flat surfaces 241 and the two second flat surfaces 242 allow a user to hold the device stably and easily.

The cap 30 includes a large-diameter section 32, a small-diameter section 33, a lid 34 and a connecting section 35. The small-diameter section 33 is disposed on a side of the large-diameter section 32 opposite to the fourth connection portion 31, and the small-diameter section 33 has a drain port 36 being selectively in communication with the interior of the tubular body 10. The lid 34 is detachably covered to the drain port 36, and the connecting section 35 is connected between the lid 34 and the large-diameter section 32. When the liquid inside the tubular body 10 is discharged through the drain port 36, it is less likely to leak out. Furthermore, when not in use, the lid 34 can be used to cover the drain port 36 to prevent the liquid (such as saliva samples, but not limited thereto) inside the tubular body 10 from leaking.

When the first collection unit 20 is used, the first collection unit 20 is screwed onto the first connection portion 13 for specimen collection, and after use, it is detached. The cap 30 is then screwed onto the second connection portion 14 to seal the opening 12, thereby preventing the specimen collected in the tubular body 10 from leaking. When the second collection unit 40 is used, the sampling member 42 of the second collection unit 40 is used to collect saliva, and the second collection unit 40 is inserted into the tubular body 10. Subsequently, the cap 30 is screwed onto the opening 12 to seal it. The specimen inside the tubular body 10 can then be discharged through the drain port 36. Therefore, the user can select either the first collection unit 20 or the second collection unit 40 for specimen collection, depending on practical requirements or user preference.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A specimen collection device, including:
a tubular body, including an opening end portion, the opening end portion including an opening communicated with an interior of the tubular body, an inner periphery wall and an outer periphery wall, the inner periphery wall having a first connection portion disposed thereon, the outer periphery wall having a second connection portion disposed thereon;
a first collection unit, detachably assembled to the opening end portion, having a third connection portion, an inlet and an outlet, the inlet communicated with the outlet, the outlet communicated with the interior of the tubular body;
a cap, detachably assembled to the opening end portion and covering the opening, having a fourth connection portion disposed thereon; and
a second collection unit, being insertable into the interior of the tubular body;
wherein one of the third connection portion and the fourth connection portion is detachably engageable with the first connection portion, and the other of the third connection portion and the fourth connection portion is detachably engageable with the second connection portion.

2. The specimen collection device of claim 1, wherein the second collection unit includes a rod and a sampling member disposed on the rod, and the sampling member is made of a deformable absorbent material.

3. The specimen collection device of claim 2, wherein the first collection unit is funnel-shaped and further includes a receiving portion and a guiding tube, the guiding tube is connected with the receiving portion and is communicated with an interior of the receiving portion, an outer periphery of the guiding tube has the third connection portion disposed thereon, the guiding tube detachably penetrates through the opening, and the third connection portion is engageable with the first connection portion.

4. The specimen collection device of claim 3, wherein the third connection portion is an external threaded structure, the first connection portion is an internal threaded structure, and the external threaded structure is screwed with the internal threaded structure.

5. The specimen collection device of claim 3, wherein an end of the guiding tube opposite to the receiving portion includes a beveled surface, and the beveled surface defines the outlet.

6. The specimen collection device of claim 1, wherein an inner surface of the cap has the fourth connection portion disposed thereon, the cap is sleeved to the opening end portion, and the fourth connection portion is engageable with the second connection portion.

7. The specimen collection device of claim 6, wherein the fourth connection portion is an internal threaded section, the second connection portion is an external threaded section, and the internal threaded section is screwed with the external threaded section.

8. The specimen collection device of claim 1, wherein the cap includes a large-diameter section, a small-diameter section, a lid and a connecting section, the small-diameter section is disposed on a side of the large-diameter section opposite to the fourth connection portion, the small-diameter section has a drain port being selectively in communication with the interior of the tubular body, the lid is detachably covered to the drain port, and the connecting section is connected between the lid and the large-diameter section.

9. The specimen collection device of claim 2, wherein the rod includes a holding section and a sampling section, a recessed section is integrally connected between the holding section and the sampling section and recessed radially inward, and the sampling member is disposed on the sampling section.

10. The specimen collection device of claim 4, wherein the first connection portion and the second connection portion correspond to each other in a radial direction of the tubular body; the receiving portion includes two first flat surfaces opposite to each other, two second flat surfaces opposite to each other and an arcuate surface, each of the two second flat surfaces is arranged between the two first flat surfaces, the arcuate surface is connected between the two second flat surfaces and has a through hole disposed therethrough, the guiding tube is connected to the arcuate surface and communicated with the through hole; an inner surface of the cap has the fourth connection portion disposed thereon, the cap is sleeved to the opening end portion, and the fourth connection portion is engageable with the second connection portion; the fourth connection portion is an internal threaded section, the second connection portion is an external threaded section, and the internal threaded section is screwed with the external threaded section; the cap includes a large-diameter section, a small-diameter section, a lid and a connecting section, the small-diameter section is disposed on a side of the large-diameter section opposite to the fourth connection portion, the small-diameter section has a drain port being selectively in communication with the interior of the tubular body, the lid is detachably covered to the drain port, and the connecting section is connected between the lid and the large-diameter section; and the rod includes a holding section and a sampling section, a recessed section is integrally connected between the holding section and the sampling section and recessed radially inward, and the sampling member is disposed on the sampling section.
